(19) United States
(12) Patent Application Publication
(10) Pub. No.: US 2007/0066602 A1
(43) Pub. Date: Mar. 22, 2007

(54) Process for Making Olanzapine Form I

(76) Inventor: Rolf Keltjens, Wijchen (NL)

Correspondence Address: SYNTHON IP INC, 7130 HERITAGE VILLAGE PLAZA, STE 202, GAINESVILLE, VA, 20155, US

(21) Appl. No.: 11/465,428

(22) Filed: Aug. 17, 2006

Related U.S. Application Data
(60) Provisional application No. 60/708,774, filed on Aug. 17, 2005.

(51) Int. Cl. A61K 31/551; C07D 498/02

(52) U.S. Cl. 514/220; 540/497

(57) ABSTRACT

Olanzapine Form I can be made by converting olanzapine benzoate to olanzapine base in an aqueous environment and isolating and drying the resulting solid olanzapine.

The present application claims the benefit of priority under 35 U.S.C. § 119(e) from U.S. provisional application Ser. No. 60/708,774, filed Aug. 17, 2005, the entire contents of which are incorporated herein by reference.

### BACKGROUND OF THE INVENTION

The present invention relates to a new method of making olanzapine in the crystalline Form I.

Olanzapine is represented by the structural formula (1) 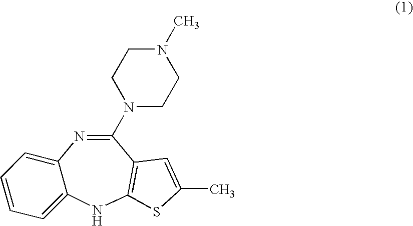
and is a pharmaceutically useful compound. In medical treatments, it is useful as an antipsychotic agent, particularly for the treatment of schizophrenia. The marketed final forms include coated tablets and quick dissolvable tablets. The single tablet comprises from 2.5 to 20 mg of olanzapine.

In the present commercially available final forms the active substance is marketed as a free base. It is a white to yellow crystalline solid that is insoluble in water (solubility at pH 6.8 is about 0.02 mg/ml).

Olanzapine and pharmaceutically acceptable salts have been suggested in EP 454436 and corresponding U.S. Pat. No. 5,229,382. In the final stage of the production process, olanzapine was obtained by a crystallization of the crude olanzapine product from acetonitrile. The patent does not refer to or identify any specific crystalline form of olanzapine.

Later, it became known that olanzapine base may exist in various crystalline modifications, including some hydrated/solvated forms, that are stable at ambient conditions (For example, see EP 733635/U.S. Pat. No. 5,736,541, WO 98-11893, and EP 831098).

The term “Form I olanzapine” was later designated in EP 733635 to the anhydrous olanzapine product that was stated to be obtainable according to the process of U.S. Pat. No. 5,229,382.

EP 733635/U.S. Pat. No. 5,736,541 disclose Form II olanzapine which is characterized by a main X-ray powder diffraction peak of d-value 10.26 A. This form has been prepared by crystallizing “technical grade” olanzapine (the product of the earlier synthesis) from ethyl acetate. This form appears to be more stable than the Form I, but it is convertible to Form I. Similarly as Form I, the Form II is an anhydrate.

U.S. Pat. No. 6,348,458 (WO 01/47933) discloses additional crystalline polymorphic forms of olanzapine, namely Form III, Form IV and Form V. These forms are made by neutralizing an acid solution of olanzapine by the addition of alkali under varying conditions to precipitate the desired olanzapine crystalline Form.

More recently, WO 03/091260 discloses Form VI olanzapine and US Appl. Publication No. 2002-0086993 discloses a polymorphic form designated as form X.

As the system used for numbering the known olanzapine forms is sometimes confusing in the prior art disclosures (for instance, the EP 828494 calls as olanzapine Form I a product that is identical with olanzapine Form II of the above definition), the “Form I” of olanzapine as used herein is defined as the solid state form of anhydrous olanzapine base which is characterized by a main peak on the X-ray powder diffraction spectrum of d-value 9.9463 A. The full diffraction pattern of the Form I has been disclosed in EP 733635. The “Form II” of olanzapine as used herein has the same definition as used in EP 733635/US 5,736,541, namely it is characterized by a main X-ray powder diffraction peak of d-value 10.26 A.

Interestingly, WO 02/18390 indicates that upon repetition of the disclosed process in U.S. Pat. No. 5,229,382, the product obtained does not correspond to the Form 1. Instead a Form II olanzapine is obtained after the crystallization from acetonitrile, while a hydrated olanzapine is obtained prior to the crystallization. The Form I complying with the above definition was actually prepared in WO 02/18390 by recrystallization of olanzapine Form II or a hydrate of olanzapine from dichloromethane, followed by drying of the wet product at 60-70° C. In fact, the product of crystallization is a dichloromethane solvate of olanzapine, which liberates dichloromethane under the conditions of drying and yields the Form I.

Furthermore, Reutzel-Edens et al. (Crystal Growth and Design, 2003, vol. 3, No. 6, 897-907) studied various solid state forms of anhydrous and hydrated forms of olanzapine. They report that while it is possible to prepare pure olanzapine Form II (which is confusingly designated as “Form I” in the article) by a direct crystallization from various solvents, it is impossible to prepare olanzapine Form I (which is designated as “Form II” in the article) in such a way. The Form I is obtainable only by a desolvation of various olanzapine solvates (methanol, dichloromethane and/or chloroform solvates) and such a product is admixed with various other forms of olanzapine. No conditions were identified that would yield pure Form I.

WO 03/97650 purports to prepare essentially pure olanzapine Form I also by a desolvation of various olanzapine solvates. However, based on the published X-ray diffraction pattern, the product appears to not be olanzapine form I as defined herein.

Essentially pure olanzapine Form I was prepared and characterized in WO 03/101997, employing a complicated purification and precipitation process.

WO 04/006933 attempts to prepare olanzapine Form I by a desolvation of various solvates and mixed solvates.

Commonly owned co-pending U.S. patent application Ser. No. 11/050,851, filed Jan. 27, 2005, relates to a process for making olanzapine Form I by heating a solid state olanzapine acetate compound to produce Form I.

Another commonly owned co-pending U.S. provisional patent application, Ser. No. 60/700717 (Atty Docket no SYN-0071PR), filed Jul. 20, 2005, relates to the formation of olanzapine Form I by the use of carbon dioxide, especially supercritical carbon dioxide, as a solvent from which the olanzapine Form I is precipitated.

The Form I olanzapine is an important product. However, it is desirable to improve the methods of making it. In particular, it is desirable to provide essentially pure olanzapine Form I, free from other polymorphic forms, by a simple and controllable process.

### SUMMARY OF THE INVENTION

The present invention relates to a process for producing crystalline olanzapine of Form I by the neutralization of olanzapine benzoate and drying of the resulting solid olanzapine product. Accordingly, an aspect of the invention relates to a process for making crystalline olanzapine Form I, which comprises reacting in an aqueous environment olanzapine benzoate with a water-soluble base to form olanzapine solids and isolating and drying the olanzapine solids to form olanzapine Form I. Suitable bases include inorganic hydroxides such as sodium hydroxide, potassium hydroxide, and ammonium hydroxide as well as a sufficiently strong organic base such as a primary, secondary or a tertiary amine. The aqueous environment is primarily water, although water miscible organic solvents can also be present. Surprisingly the dried olanzapine base can yield Form I (anhydrate) olanzapine in good yield and purity.

### DETAILED DESCRIPTION OF THE INVENTION

The present invention relates to the discovery that olanzapine Form I can be formed by converting olanzapine benzoate to olanzapine base (i.e., a neutralization of the olanzapine salt) in an aqueous environment and then drying the isolated olanzapine base. The solid olanzapine initially produced by the reaction, e.g., as isolated wet cake, appears to contain water similar to dihydrate Form B olanzapine. But drying of the isolated olanzapine solids apparently converts whatever hydrate is present to Form I. That Form I is produced instead of Form II or some hydrate form is surprising given that water is present and the results of the solvate and hydrate conversions described in the above-mentioned patents and articles would suggest otherwise. The process is fairly robust and yields Form I olanzapine under a variety of conditions, as will be discussed below, and in good yields and purity, both crystallographic purity and non-olanzapine molecule purity.

The process comprises reacting olanzapine benzoate and a water-soluble base in an aqueous environment. The “aqueous environment” means that liquid water is present to a significant extent, generally at least 50%, typically at least 80% and more typically at least 99% of the liquid reaction medium. The liquid medium is preferably water per se, but water miscible organic liquids such as lower alcohols, aliphatic ketones, ethers such as dioxan or tetrahydrofuran may be present as well.

Olanzapine benzoate, which is described in commonly owned U.S. patent application Ser. No. 11/050,852, filed Jan. 27, 2005, is sparingly soluble in water. It can be introduced or provided to the aqueous environment by any suitable method. Further, it is not necessary that the olanzapine benzoate is fully dissolved in the aqueous environment. Instead, the olanzapine benzoate can be a slurry; i.e., solid olanzapine benzoate in a liquid aqueous medium. However, low concentrations of olanzapine benzoate could be entirely dissolved to form a solution, if desired, especially if organic solvents are present to increase the solubility of the olanzapine benzoate. In any event, a slurry has been found to work well. Methods of making the olanzapine benzoate are described in the above-mentioned U.S. application Ser. No. 11/050,852, all of which can be used in the present invention.

The olanzapine benzoate is converted to olanzapine by reaction with a water soluble base. A “water soluble base” means a base having a water solubility of at least 1 mg, preferably at least 5 mg, per 1 ml of water. The water solubility can both facilitate a more efficient reaction and enhance the removal of the base from the olanzapine solids after the reaction by washing the isolated solids, e.g., wet cake, with water. Generally the base is an inorganic hydroxide or an organic amine. Examples of such hydroxides are sodium hydroxide, potassium hydroxide, and ammonium hydroxide. Examples of amines include triethylamine and pyridine. Typically amines are not used for environmental reasons. The co-product of the reaction is a benzoate salt of the base used in the neutralization and such a salt is preferably sufficiently soluble in the medium so as to be readily separated from the olanzapine solids. For example, the benzoate salt may have sufficient solubility so as to not precipitate from the aqueous medium and/or to be easily separable from the olanzapine solids by washing, e.g., by washing the (wet) cake with water or other aqueous solution.

In order to carry out the reaction, the olanzapine benzoate and water soluble base are combined in an aqueous environment. How this is achieved, including the rate and order of the contact, is not particularly limited and any suitable procedure can be used. For instance, the base may be added in form of a solid or a solution to a solution and/or slurry of olanzapine benzoate. Alternatively a solution and/or suspension of olanzapine benzoate may be added to the base in solid and/or dissolved form. The solvents in each of the aforementioned solutions and slurries is typically water although other organic solvents can be present so long as the total amount of water present in the liquid reaction media, once the reaction starts, is at least 50% of all of the solvents, as described above. The water soluble base and the olanzapine benzoate can be combined all at once or over time in continuous or discrete portions. The molar amount of the base is generally at least equal to the molar amount of olanzapine benzoate and can be present in substantial molar excess. For instance, the water soluble base can be up to 10 fold molar excess of the olanzapine benzoate (i.e. 10 moles base to 1 mole olanzapine benzoate), and generally is 1.1 to 5 molar excess.

Typically the olanzapine benzoate is combined as an aqueous slurry containing water and no more than 20% organic solvent(s), preferably no more than 10% organic solvent(s), and typically 0% organic solvents. The amount of olanzapine benzoate is not particularly limited. A convenient and/or practical amount generally falls within the range of 5-100 grams of olanzapine benzoate per 1 liter of liquid media, e.g. per 1 liter of water. Other amounts can also be used. The water soluble base is typically combined as an aqueous solution. For convenience and practicality, the base is relatively concentrated such as in the range of 0.5N to 10 N, more typically about 1 to about 5 N. Using less concentrated base simply increases the amount of water which in large scale can be inconvenient. In one embodiment, the aqueous solution of the water soluble base, preferably sodium hydroxide is added, generally in one portion, into the aqueous slurry of olanzapine benzoate.

Once combined, the reaction takes place in the aqueous environment and the olanzapine benzoate is converted to olanzapine solids. It is not necessary that the reaction ever provides a complete solution, e.g. a solid phase can always be present, in order to be effective. In fact, it is generally preferred that the reaction proceeds as a slurry with the solid changing from olanzapine benzoate to olanzapine base. Without wishing to be bound by theory, the reaction presumably takes place in solution; e.g. the small amount of olanzapine benzoate that dissolves into the liquid reaction media reacts with the base to form olanzapine. Because olanzapine is less water soluble than olanzapine benzoate, the olanzapine precipitates more readily from the aqueous environment. The loss in dissolved olanzapine benzoate by this conversion and precipitation allows more of the solid olanzapine benzoate to be dissolved, which in turn allows the base to react with the olanzapine benzoate to form olanzapine which is precipitated, etc., until all of the olanzapine benzoate is converted to olanzapine. In any event and regardless of the precise mechanism, the combining in an aqueous environment of the base with olanzapine benzoate, even in slurry form, produces olanzapine base in solid form, that is “olanzapine solids.” The olanzapine solids are generally particulates and of largely crystalline form. Some if not all of the olanzapine solids are an apparently novel hydrate crystalline form of olanzapine. This form is similar to Form B, but unlike Form B, this form does not convert to the anhydrous Form II. Instead, this hydrate converts to anhydrous Form I.

Without wishing to be bound by any theory, it is thought that the low water solubility of the olanzapine benzoate contributes to the mechanism of forming these precursor olanzapine Form I crystals and/or olanzapine Form I itself. Thus it is possible that other sparingly water soluble salts as described in the U.S. application Ser. No. 11/050,852 may also be suitable salts for forming olanzapine Form I by an analogous reaction process in an aqueous environment.

The reaction time is not particularly limited with completion of the reaction typically being achieved in 2 hours or less, although longer completion times are also possible. The reaction, especially when a slurry is used, may be carried out with stirring, which includes agitation and/or mixing as well, in order to enhance the reaction times and/or yields.

The reaction temperature is also not particularly limited and typically falls within the range of 0° C. to 80° C., although higher or lower temperatures may be used. More specifically, when seeding crystals of olanzapine Form I are not used, the reaction temperature is generally low, typically 5° C. or less, in order to avoid the formation of undesired crystalline forms. When using seeds during the reaction, the temperature can be much higher without any apparent change in morphology. The seeds are typically added to the olanzapine benzoate in the reaction media prior to contact with the water soluble base, although later addition is also possible. Generally the amount of olanzapine Form I seed is within the range of 1 to 10 wt % of the amount of olanzapine benzoate. The use of seeds is generally helpful, though not required.

Once the reaction has occurred, the olanzapine solids are formed, presumably by precipitation as theorized above. The precipitation is generally spontaneous and may be concurrent with the reaction depending upon the nature of the aqueous environment and the concentrations involved. However, if necessary, the olanzapine solids can be precipitated, or more completely precipitated, by using known precipitation techniques such as reducing the volume of the liquid, which may be especially useful when organic solvents are also present, dropping the temperature, adding a contra-solvent such as water, and/or adding seeding crystals of olanzapine Form I as described above.

After the reaction, the olanzapine solids are isolated and dried to form olanzapine Form I. The isolation generally involves separating the olanzapine solids from the liquid medium by any suitable technique; typically by filtration or centrifugation. Typically the isolated product at this point is a wet cake. If desired, the wet cake can be washed, such as with water, to remove any unreacted base and/or co-products (e.g., sodium benzoate) therefrom. Thus, it is not necessary that any excess base be neutralized prior to the separation as such can be removed by a water wash of the cake. Nevertheless, a neutralization of all or part of the excess water soluble base prior to, e.g., filtration, may be performed if desired by adding a suitable acid that forms a soluble salt (e.g. hydrochloric acid). Somewhat surprisingly, the olanzapine solids do not readily undergo solvent mediated polymorphic transformations even if not separated from the reaction media for a period of time; i.e., up to 20 hours or more after the complete addition of the base. The separation technique, such as filtration or centrifugation normally proceeds at ambient temperature as does the optional washing step(s).

The isolated olanzapine solids, e.g., the optionally washed wet cake, is then dried to provide olanzapine Form I. The drying process is believed to convert a hydrated form of olanzapine into the desired anhydrate Form I. However, it could be that the isolated olanzapine solids already contain olanzapine Form I either exclusively with un-bound water or as a mixture with other olanzapine forms. In the later case, the drying is thought to convert the crystal to olanzapine Form I. Whether a true crystalline conversion occurs or water is simply driven off, the drying is considered to “form” the olanzapine Form I from the isolated solids for purposes of the present invention. The drying process is not particularly limited and may proceed at ambient or diminished pressure and at a temperature from 20 to 60° C. Typically higher temperatures, such as 50-60° C. are preferred as are sub-ambient pressures. Commercially available vacuum ovens are suitable for drying the olanzapine solids.

The product may be homogenized by milling, sieving etc., and can be used as the active drug substance in various pharmaceutical formulations for treating psychoses.

The isolated and dried olanzapine Form I is generally pure or substantially pure of other substances and is typically, though not necessarily, at least 97%, and usually at least 99% pure olanzapine. The olanzapine product should contain less than 1% and preferably less than 0.1% of any organic volatile impurities; e.g., organic solvents, triethylamine, etc. The isolated and dried olanzapine is also generally morphologically pure Form I olanzapine; e.g., at least 90% Form I olanzapine, preferably at least 95% Form I, more preferably at least 99% Form I, and most preferably essentially 100% Form I olanzapine, based on the total weight of crystalline olanzapine. Practically speaking, the Form I olanzapine produced by the present invention preferably shows no indication of Form II olanzapine, and more preferably no indication of any other Form of olanzapine, under x-ray powder diffraction analysis.

The invention will be further described with reference to the following non-limiting examples.

### EXAMPLES

### Example 1

To a suspension of 1.0 g olanzapine benzoate in 30 ml of water was added, at a stirring speed of 500 rpm and in one portion, 10 ml of 1N aqueous NaOH at 4° C. No seeding. After 30-40 minutes of stirring, the solid material was isolated by filtration at said temperature, dried at the air and subsequently at 50° C. in vacuo overnight.
Yield=0.68 g (94%)
¹H-NMR: no benzoic acid present
XRPD: Form I

### Example 2

The same as in Example 1, but before addition of aqueous 1N NaOH, 100 mg of seeds of Form I were added in one portion.
Yield=0.81 g (98%), corrected for amount of seeds
¹H-NMR: no benzoic acid present
XRPD: Form I

### Example 3

The same as in Example 2, but 15 ml water and 30 ml aqueous 1N NaOH was used
Yield=0.83 g (100%), corrected for amount of seeds
¹H-NMR: no benzoic acid present
XRPD: Form I

### Example 4

The same as in example 2, but isolated after 2 hours 15 minutes.
Yield=0.81 g (99%), corrected for amount of seeds
XRPD: Form I
DSC/TGA: no hydrates/water present

### Example 5

The same as example 2, but isolated after 4hours 15 minutes.
Yield=0.79 g (96%), corrected for amount of seeds
   XRPD: Form I
   DSC/TGA: no hydrates/water not present

### Example 6

The same as example 2, but isolated after 20 hours.
Yield=0.79 g (96%), corrected for amount of seeds
   XRPD: Form I
   DSC/TGA: no hydrates/water not present

### Example 7

To a suspension of 2.0 g of olanzapine benzoate and 200 mg seeds of olanzapine Form I in 30 ml of water was added, at a stirring speed of 500 rpm in one portion, 10 ml of 2N aqueous NaOH at 4° C. After stirring for 4 hours at 4° C., the solid material was isolated by filtration, washed with a small amount of water, dried overnight at 50° C. in vacuo.
Yield=1.6 g (98%), corrected for amount of seeds
DSC/TGA: no hydrates/water not present

### Example 8

The same as in example 7, but only 0.100 g of seeds of olanzapine Form I.
Yield=1.52 g (99%), corrected for amount of seeds
DSC/TGA: no hydrates/water not present

### Example 9

The same as in example 7, but only 0.050 g of seeds of olanzapine Form I.
Yield=1.48 g (99%), corrected for amount of seeds
XRPD: only olanzapine free base Form I
DSC/TGA: no hydrates/water not present

### Example 10

The same as in example 7, but only 0.020 g of seeds of olanzapine Form I.
Yield=1.45 g (99%), corrected for amount of seeds
XRPD: only olanzapine free base Form I
DSC/TGA: no hydrates/water not present

### Example 11

To a suspension of 10.0 g of olanzapine benzoate and 1.0 g of seeds of olanzapine Form I in 300 ml of water was added slowly at a stirring speed of 200 rpm (mechanical stirrer) 100 ml 1N aqueous NaOH at 4° C. After stirring for 2 hours at 4° C., the solid material was isolated by filtration, washed with 80 ml of water, dried overnight at 60° C. in vacuo.
Yield=8.1 g (99%), corrected for amount of seeds
XRPD; Form I, very small crystals

### Example 12

To a suspension of 100.0 g of olanzapine benzoate and 5.0 g of seeds of olanzapine Form I in 2000 ml of water was added slowly (addition time 20 minutes) at a stirring speed of 300 rpm (mechanical stirrer) 1000 ml of pre-cooled 1N aqueous NaOH at 4° C. After stirring for 2 hours at 4° C., the solid material was isolated by filtration, washed with 500 ml of water, and dried overnight at 60° C. in vacuo.
Yield=˜81 g (99%)
XRPD; Form I olanzapine
Purity (HPLC): 99.9%
Assay (HPLC): 99.4% (m/m)
Total impurities: 0.07% (m/m)
Highest single impurity: 0.05% (m/m)
Residual Solvents: ethyl acetate (<0.01%)
Water content (KF): 0.1%
Ion chromatography: Na⁺: 0.02%
Benzoate (HPLC): 0.05% (m/m)

### Example 13

To a suspension of 1.0 g of olanzapine benzoate and 100 mg of seeds of olanzapine Form I in 30 ml of water was added at a stirring speed of 500 rpm in one portion 10 ml of 1N aqueous NaOH at 4° C. After 2 hours, the solid material was isolated by filtration, washed with a small amount of water, and dried overnight at 50° C. in vacuo.
Yield=0.81 g (99%), corrected for amount of seeds
XRPD: Form I olanzapine

### Example 14

The same as in example 13, but reaction temperature 20° C.
Yield=0.81 g (99%), corrected for amount of seeds
XRPD: Form I olanzapine

### Example 15

The same as in example 13, but reaction temperature 40° C.
Yield=0.80 g (98%), corrected for amount of seeds
XRPD: Form I olanzapine

### Example 16

The same as in example 13, but reaction temperature 60° C.
Yield=0.80 g (98%), corrected for amount of seeds
XRPD: Form I olanzapine

### Example 17

To a suspension of 2.0 g of olanzapine benzoate and 200 mg of seeds of olanzapine Form I in 30 ml water was added at a stirring speed of 500 rpm in one portion 10 ml of 2N aqueous NaOH at 60° C. After 1 hour, the solid material was isolated by filtration, washed with a small amount of water, and dried overnight at 50° C. in vacuo.
Yield=1.6 g
XRPD: Form I olanzapine

### Example 18

The same as in example 17, but reaction temperature 80° C.
Yield=1.6 g
XRPD: Form I

Each of the patents, patent applications, articles, and publications mentioned above is incorporated herein by reference in its entirety. The invention having been thus described, it will be obvious to the worker skilled in the art that the same may be varied in many ways without departing from the spirit of the invention and all such modifications are included within the scope of the present invention as set forth in the following claims.

We claim:

1. A process for making olanzapine Form I, which comprises:
reacting in an aqueous environment olanzapine benzoate with a water-soluble base to form olanzapine solids;
isolating and drying said olanzapine solids to form olanzapine Form I.

2. The process according to claim 1, which further comprises forming an aqueous slurry of olanzapine benzoate and adding said water-soluble base thereto to carryout said reaction step.

3. The process according to claim 2, which further comprises adding olanzapine Form I seeds to said aqueous slurry of olanzapine benzoate prior to said addition of said water-soluble base.

4. The process according to claim 2, wherein said water-soluble base is in the form of an aqueous solution when added to said aqueous slurry.

5. The process according to claim 4, wherein said aqueous slurry comprises no more than 20% organic solvent.

6. The process according to claim 5, wherein said aqueous slurry comprises no organic solvent.

7. The process according to claim 1, wherein said water soluble base is selected from the group consisting of sodium hydroxide, potassium hydroxide, ammonium hydroxide, and combinations thereof.

8. The process according to claim 2, wherein said water soluble base is selected from the group consisting of sodium hydroxide, potassium hydroxide, ammonium hydroxide, and combinations thereof.

9. The process according to claim 1, wherein the temperature during said reaction is within the range of 0 to 80° C.

10. The process according to claim 1, wherein the temperature during said reaction is 5° C. or less.

11. The process according to claim 1, wherein said olanzapine Form I is at least 99% pure.
